(19) United States
(12) Patent Application Publication
(10) Pub. No.: US 2022/0098157 A1
(43) Pub. Date: Mar. 31, 2022

(54) COMPOUNDS WITH AT LEAST ONE CYCLIC UREA MOIETY AND THEIR USE IN CORROSION PREVENTION

(71) Applicant: NOURYON CHEMICALS INTERNATIONAL B.V., ARNHEM (NL)

(72) Inventors: Ina EHLERS, Stenungsund (SE); Eike Nicolas KANTZER, Uddevalle (SE); Rolf Krister EDVINSSON, Partille (SE); Karl Fredrik LAKE, Södertälje (SE); Hendrik VAN DAM, Ede (NL); Antoon Jacob Berend TEN KATE, Arnhem (NL); Michiel Jozef Thomas RAAIJMAKERS, Deventer (NL); Rens VENEMAN, Amersfoort (NL); Slavisa JOVIC, Utrecht (NL); Lawien Feisal ZUBEIR, Deventer (NL)

(73) Assignee: NOURYON CHEMICALS INTERNATIONAL B.V., ARNHEM (NL)

(21) Appl. No.: 17/310,498

(22) PCT Filed: Feb. 4, 2020
(86) PCT No.: PCT/EP2020/052776

§ 371 Date: Aug. 6, 2021

(30) Foreign Application Priority Data
Feb. 7, 2019 (EP) 19155972.3

(51) Int. Cl. C07D 233/34; C09K 15/30; C23F 11/14

(52) U.S. Cl. CPC C07D 233/34; C23F 11/149; C09K 15/30

(57) ABSTRACT

A compound comprises at least one cyclic urea moiety including a head portion and a tail portion connected through an amide moiety or a cyclic amidine moiety, wherein the head portion comprises a polyalkylenepolyamine structure comprising 3 to 10 C2-C4 alkylene moieties present between nitrogen atoms, wherein the alkylene moieties may be the same or different and may be substituted with one or more C1 to C3 alkyl groups, wherein at least one of the alkylene moieties together with its adjoining nitrogen atoms is in the form of a cyclic alkylene urea moiety of the formula

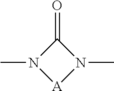

wherein A is a C2 to C4 alkylene moiety which may be substituted with one or more C1 to C3 alkyl groups and wherein the tail portion includes an alkyl or alkenyl group with 6 to 24 carbon atoms which may be substituted.

### CROSS-REFERENCE TO RELATED APPLICATION

This application is a U.S. National-Stage entry under 35 U.S.C. § 371 based on International Application No. PCT/EP2020/052776, filed Feb. 4, 2020, which was published under PCT Article 21(2) and which claims priority to European Application No. 19155972.3, filed Feb. 7, 2019, which are all hereby incorporated in their entirety by reference.

### TECHNICAL FIELD

The present disclosure pertains to compounds with at least one cyclic urea moiety and their use in corrosion prevention.

### BACKGROUND

Corrosion generally is a destructive attack on metal involving oxidation or other chemical attack. Corroded metal components can have reduced functionality, causing inefficiencies or inoperability of the industrial process. Corroded components may need to be discarded, repaired or replaced. In addition, corrosion products accumulate and may decrease the rate of heat transfer between the corroded material and the water or other fluid media. Therefore, corrosion may reduce the efficiency of the system operation where efficient cooling is a factor. Corrosion of metallic components in industrial plants can cause system failures and even plant shutdowns.

Corrosion prevention additives are used in many of applications where surfaces, which are sensitive to corrosion, in particular metal surfaces, are exposed to conditions which promote corrosion, generally exemplified by the presence of water and oxygen, and often by the presence of further corrosion-exacerbating components such as, for example, organic and inorganic acids and bases, CO₂, hydroxides, sulphur-containing compounds such as SOₓ, H₂S, hydrosulphide compounds, Sₓ²⁻, NOₓ compounds, and salts.

Traditional uses of corrosion inhibitors include, for example, coatings, water treatment applications, metal cutting fluids, applications in lubricating oils and fuels, and in the oil and gas industry, for example in the protection of metal surfaces, preferably ferrous metals or alloys, such as iron and steel, of pipelines, pumps, tanks and other equipment. The corrosion inhibitors of the present disclosure may be used in all applications where corrosion inhibitors are used conventionally.

In the past, compounds based on metals, such as lead and chromate have been used as corrosion inhibitors, but their use is restricted in view of HSE considerations. In consequence, organic compounds with anti-corrosive properties are being developed.

U.S. Pat. No. 5,746,946 describes a class of compounds of the formula

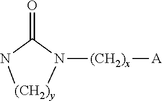

wherein A is —NH—C(O)—CHR1-CHR2-COOH, —O—C(O)—CHR1-CHR2-COOH, —NH—C(O)—CHR1, and —O—C(O)—CHR1, or a moiety of the formula

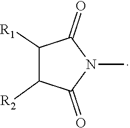

It has been found that while some compounds described in U.S. Pat. No. 5,746,946 have attractive anticorrosive performance, there is need in the art for compounds with improved anticorrosive performance. The present disclosure provides such compounds.

It is noted that U.S. Pat. No. 3,312,619 describes 2-substituted imidazolidines useful as detergents in lubricating oil compositions.

### BRIEF SUMMARY

This disclosure provides a compound comprising at least one cyclic urea moiety and comprising a head portion and a tail portion connected through an amide moiety or a cyclic amidine moiety, wherein the head portion comprises a polyalkylenepolyamine structure comprising about 3 to about 10 C2-C4 alkylene moieties present between nitrogen atoms, wherein the nitrogen atom in the amide moiety or cyclic amidine moiety is counted as a nitrogen atom of the polyalkylenepolyamine structure, wherein the alkylene moieties may be the same or different and may be substituted with one or more C1 to C3 alkyl groups, wherein at least one of the alkylene moieties together with its adjoining nitrogen atoms is in the form of a cyclic alkylene urea moiety of the formula:

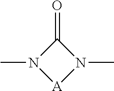

wherein A is a C2 to C4 alkylene moiety which may be substituted with one or more C1 to C3 alkyl groups and wherein the tail portion comprises an alkyl or alkenyl group with about 6 to about 24 carbon atoms which may be substituted.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description.

The present disclosure pertains to compounds with at least one cyclic urea moiety consisting of or including a head portion and a tail portion connected through an amide moiety or a cyclic amidine moiety, wherein the head portion comprises a polyalkylenepolyamine structure comprising about 3 to about 10 C2-C4 alkylene moieties present between nitrogen atoms, the nitrogen atom in the amide moiety or cyclic amidine moiety being counted as a nitrogen atom of the polyalkylenepolyamine structure, wherein the alkylene moieties may be the same or different and may be substituted with one or more C1 to C3 alkyl groups, wherein at least one of the alkylene moieties together with its adjoining nitrogen atoms is in the form of a cyclic alkylene urea moiety of the formula

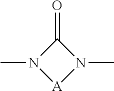

wherein A is a C2 to C4 alkylene moiety which may be substituted with one or more C1 to C3 alkyl groups and wherein the tail portion consists of or includes an alkyl or alkenyl group with about 6 to about 24 carbon atoms which may be substituted.

It has been found that compounds as contemplated herein show improved anticorrosive performance as compared to the compounds described in U.S. Pat. No. 5,746,946. Further advantages of the imidazolidinone compounds and specific embodiments and applications thereof will become apparent from the further specification.

In one embodiment the present disclosure pertains to a compound comprising at least one cyclic urea moiety, the compound consisting of or including a head portion and a tail portion connected through a linking moiety, wherein the head portion and the tail portion have a different structure, as will be discussed in more detail below. This asymmetric structure is necessary for the anticorrosive effect of the present disclosure.

The compound of the present disclosure has a head portion comprising a polyalkylenepolyamine structure comprising about 3 to about 10 alkylene moieties present between nitrogen atoms. To avoid excessively large molecules the head portion comprises at most about 10 alkylene moieties present between nitrogen atoms. It may be particularly preferred for the head portion to comprise about 3 to about 8 alkylene moieties, more in particular about 3 to about 6 alkylene moieties.

The polyalkylenepolyamine structure in the head portion of the anticorrosive amide compound may be straight-chain or branched. In addition to at least one alkyleneurea moiety, it may comprise one or more moieties of the formula

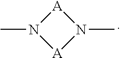

The alkylene moieties may be the same or different. It is preferred for the alkylene moieties to be selected from propylene moieties, isopropylene moieties, and ethylene moieties. It may be preferred for the alkylene moieties to be ethylene moieties.

The head portion of the compounds as contemplated herein contains at least one cyclic alkylene urea moiety of the formula

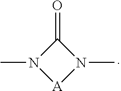

Depending on the number of alkylene moieties present between nitrogens in the head portion of the molecule, the head portion may contain more than one, e.g., two, three, four, five, six, or even more alkylene urea moieties. In general, it is preferred for the head portion to contain one to six, in particular one to four alkylene urea moieties.

The head portion of the compound as contemplated herein may or may not comprise one or, in the case of branched structures, more than one terminal primary amine groups. This group may have a dual effect. On the one hand, the primary amine group may lead to increased interaction with the substrate, leading to increased corrosion inhibition. On the other hand, the presence of a terminal amine group during the manufacture of the compound as contemplated herein may lead to the formation of side products containing a polyalkylenepolyamine structure surrounded by two or more tail groups. These compounds, which do not consist of alkylene urea moieties consisting of or including a head and a tail group as required by the present disclosure do not show the desired anticorrosive effect to the fullest possible extent.

Accordingly, the presence of terminal primary amine groups may be attractive, as long as the presence of compounds containing more than one tail group can be prevented.

The compounds used in the present disclosure comprise a head portion and a tail portion connected through an amide moiety or a cyclic amidine moiety.

In one embodiment the head portion is connected to the tail portion through an amide moiety.

In one embodiment the head portion is connected to the tail portion through a cyclic amidine moiety. The cyclic amidine moiety may be regarded as the cyclic amidine condensation products of the amide compounds described herein. As will be clear to the skilled person, a —NH-A-NH—CO—R structure can condense under the removal of water to form a cyclic structure for the formula

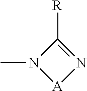

It may be preferred for R1 to be an optionally substituted alkyl or alkenyl group with about 4-about 18 carbon atoms and R2 to be hydrogen or an optionally substituted alkyl or alkenyl group with about 1-about 18 carbon atoms. In one embodiment R1 is isobutyl or polyisobutenyl. In one embodiment R1 is isobutyl or polyisobutenyl and R2 is hydrogen. For substitutions reference is made to what is stated below on the substitutions for R.

In one embodiment, the head portion of the compound as contemplated herein consists of or includes a polyalkylenepolyamine structure, in particular a polyethylenenepolyamine structure. Examples of amide compounds as contemplated herein which have a head group with a preferred polyethylenenepolyamine structure are given in the formulas 1 to 6 below.

Formula 7 and 8 show examples of compounds which contain a cyclic amidine moiety, in this case an imidazoline moiety, as linker. These compounds may be regarded as condensation products of the corresponding amide compounds. Formula 7 shows the condensation product of the compound of formula 1 and formula 8 shows the condensation product of the compound of formula 5.

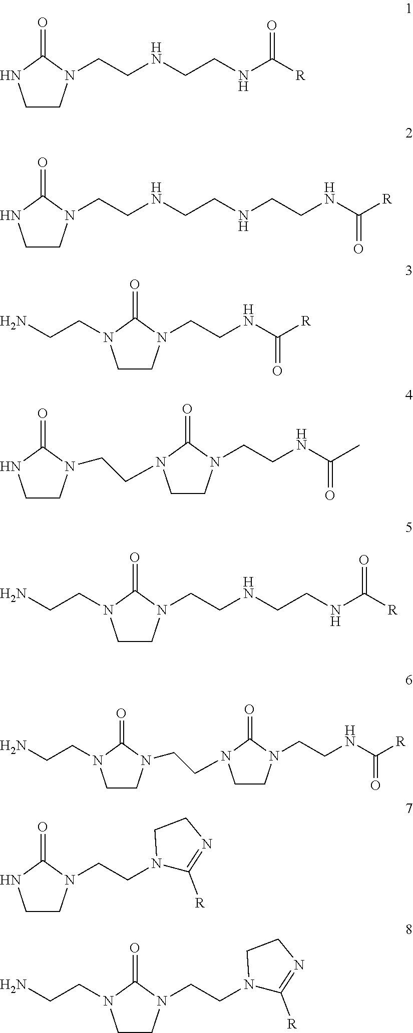

The tail portion of the compounds of the present disclosure is intended to provide surfactant properties to the compound of the present disclosure by acting as a hydrophobic tail to the relatively hydrophilic cyclic urea-containing head group, resulting in effective anticorrosive properties. For example, where the anticorrosive compound is added to an aqueous medium the tail portion of the compound of the present disclosure is intended to participate in forming a hydrophobic layer on the substrate to help to reduce or prevent polar compounds such as water to reach the surface. This contributes to the anticorrosive properties of the compounds.

The tail portion of the compound as contemplated herein, e.g., R in formulas 1-8 above, consists of or includes an optionally substituted alkyl or alkenyl group with at least about 6 carbon atoms. In one embodiment, the tail portion consists of or includes an optionally substituted alkyl or alkenyl group with about 6-about 24 carbon atoms, in particular straight-chain alkyl or alkenyl groups with about 6-about 20 carbon atoms, optionally substituted with groups selected from one to three C1-C3 alkyl groups, one to three hydroxyl groups, preferably one or two, one or two carboxylic acid groups, preferably one, and one or two amine groups, preferably one. The tail portion is preferably relatively hydrophobic in nature. It is therefore preferred for it to be substituted with at most three groups selected from hydroxyl groups, amine groups and carboxylic acid groups, in particular at most two, more in particular at most one, still more in particular none.

In one embodiment of the present disclosure, the tail portion corresponds to the tail of a fatty acid selected from the group octanoic acid, nonanoic acid, decanoic acid, undecanoic acid, undecylenic acid, dodecanoic acid, tridecanoic acid, tetradecanoic acid, hexadecanoic acid, octadecanoic acid, oleic acid, and mixtures thereof.

Commercially available fatty acid mixtures may be used in the present disclosure, e.g., fatty acids derived from tallow, fatty acids derived from tall oil, fatty acids derived from coco-oil, fatty acids derived from palm oil, etc.

The compounds according to the present disclosure can be manufactured as desired. In one embodiment the amide compounds are manufactured by reacting a polyalkylenepolyamine structure comprising about 3 to about 10 alkylene moieties as defined above present between nitrogen atoms and at least one alkylene urea moiety as defined above, the polyakylenepolyamine structure having a primary amine group with a fatty acid of the formula HOOC—R, wherein the R group corresponds to the tail portion of the amide as contemplated herein. This reaction results in the formation of the amide bond connecting the head portion and the tail portion of the amide as contemplated herein. This reaction can be carried out at a temperature in the range of from about 100-about 250° C. As amide formation reactions of this type are known in the art, they require no further elucidation here.

The cyclic amidine condensation product, which contains a cyclic structure of the formula

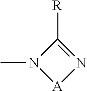

can be obtained from the amide compound by removal of water. Manufacture of the condensation product can, for example, be affected by carrying out the reaction step in the amide formation under such conditions that additional water is removed, e.g., by the application of a vacuum. Condensation reactions of this type are known in the art and require no further elucidation here.

The polyalkylenepolyamine structure comprising about 3 to about 10 alkylene moieties as defined above present between nitrogen atoms and at least one alkylene urea moiety as defined above can be prepared by reacting a polyalkylenepolyamine structure comprising at least about 3 alkylene moieties as defined above present between nitrogen atoms with a carbon oxide delivering agent. The carbon oxide delivering agent can in particular be selected from CO2 and organic compounds in which a carbonyl moiety is available including urea, and derivatives thereof; linear and cyclic alkylene ureas, especially cyclic urea, mono or di-substituted alkylene ureas, alkyl and dialkyl ureas, linear and cyclic carbamates, organic carbonates and derivatives or precursors thereof. In general, the compounds of the present disclosure can be prepared by combining the polyalkylenepolyamine structure with the carbon oxide delivering agent, and allowing the compounds to react in the liquid phase. Suitable temperatures and pressures will depend on the carbon oxide delivering agent used and will vary between about 20 and about 300° C., with the pressure being selected such that the reaction mixture is in the liquid phase.

The amount of anticorrosive compound to be used in the prevention of corrosion will depend on the use in question, be it in a coating, drilling fluid, water treatment fluid, or any other use. As a general range, the dosage of corrosion inhibitor needed to obtain a sufficient protection varies with the application, but is suitably dosed in such an amount that the concentration at the point of protection is between about 0.1 to about 50,000 ppm (i.e., from about 0.00001 to about 5 wt %), or more specifically in the range of about 1 to about 500 ppm, or about 1 to about 200 ppm, or about 1 to about 100 ppm, based on the amount of the composition which is in contact with the substrate, e.g., the coating composition or the drilling fluid.

In industrial processes, water-containing fluids are used in many applications and they are often in contact with metal parts under conditions that promote corrosion. Some examples are fluid compositions containing water that are being processed in reactors, heat exchangers, pumps or valves, that are transported and stored in pipes and tanks or that are used as cleaning and cooling fluids. The addition of corrosion inhibitors to these compositions is one embodiment of the present disclosure. In this case, not wishing to be bound by theory, it is believed that the corrosion inhibitors of the present disclosure work by interacting with the surfaces, and self-assemble on the surface to form a film thereon. Thereby, corrosion inhibitors form a protective layer on the metal surface which prevents the corrosive substances from reaching the surface and, thus, stop or reduce the corrosion process.

The present disclosure will be elucidated by the following examples, without being limited thereto or thereby.

### Example 1

An amide as contemplated herein was prepared starting from cyclic 1,3-diurea tetraethylene pentamine A comparative amide was prepared from the cyclic urea derivative of diethylenetriamine, as shown in the scheme below

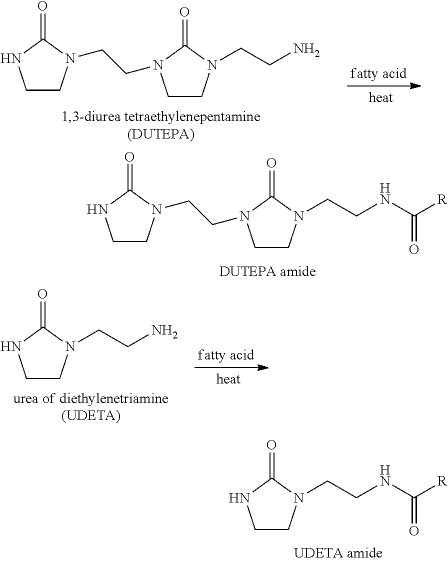

Amide synthesis was performed by reaction of the cyclic urea polyethyleneamines with fatty acids in a molar ratio of 1:1. Tall oil fatty acids (TOFA), specifically Sylfat 2 from Kraton, were used as fatty acid in both examples. TOFA mainly comprise palmitic acid (C16:0), oleic acid (C18:1) and linoleic acid (C18:2). The cyclic urea polyethyleneamine was placed in a round bottom flask equipped with stirrer and the fatty acid was slowly added. After complete addition, the reaction mixture was heated to 150° C. for 2 h followed by 1 h at 190° C. Completion of the reaction was controlled by measuring the acid value of the product by titration.

Corrosion inhibition performance was measured in an acid immersion weight loss test based on guidelines of the ASTM G31-72 protocol. The test compound was dissolved in ethanol (0.5 wt % solution) and from this standard solution a certain amount was added to 15 wt % HCl to reach the test concentration of 10 ppm. The solution was stirred continuously and maintained at 50° C. during the test period. Three metal coupons were placed in the solution and corrosion was determined by measuring weight loss after 6 h of immersion. Corrosion inhibition performance is stated as average of three replicates and calculated relative to corrosion of metal coupons in HCl solution without addition of an inhibitor, as stated below.

% corrosion inhibition=[1−(mass loss with inhibitor/mass loss without inhibitor)]×100

The results are presented in Table 1 below.

TABLE 1

| Inhibitor (10 ppm) | Corrosion inhibition |
|---|---|
| Comparative - UDETA amide | 81% |
| Disclosure - DUTEPA amide | 87% |

As can be seen from Table 1, the amide as contemplated herein showed improved corrosion inhibition as compared to the comparative amide.

### Example 2

A mixture of cyclic urea polyethyleneamines was reacted with fatty acids in a molar ratio of 1:1 as described in example 1. Tall oil fatty acids (TOFA), specifically Sylfat 2 from Kraton, were used as fatty acid. The resulting composition comprised 50 wt. % amine of formula 2 above, 10 wt. % of the amide of formula 5 above, and 10 wt. % of the amide of formula 6 above. The remaining 30 wt. % of the composition was not identified. The anticorrosive properties of the composition were determined as described in Example 1. A corrosion inhibition of 85% was found.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. Compound comprising at least one cyclic urea moiety and comprising a head portion and a tail portion connected through an amide moiety or a cyclic amidine moiety, wherein the head portion comprises a polyalkylenepolyamine structure comprising about 3 to about 10 C2-C4 alkylene moieties present between nitrogen atoms, wherein the nitrogen atom in the amide moiety or cyclic amidine moiety is counted as a nitrogen atom of the polyalkylenepolyamine structure, wherein the alkylene moieties may be the same or different and may be substituted with one or more C1 to C3 alkyl groups, wherein at least one of the alkylene moieties together with its adjoining nitrogen atoms is in the form of a cyclic alkylene urea moiety of the formula: 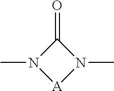
wherein A is a C2 to C4 alkylene moiety which may be substituted with one or more C1 to C3 alkyl groups and wherein the tail portion comprises an alkyl or alkenyl group with about 6 to about 24 carbon atoms which may be substituted.

2. Compound according to claim 1, wherein the head portion comprises about 3 to about 8 alkylene moieties present between nitrogen atoms.

3. Compound according to claim 1, wherein the alkylene moieties are selected from propylene moieties, isopropylene moieties and ethylene moieties.

4. Compound according to claim 1 wherein the head portion is connected to the tail portion through an amide moiety.

5. Compound according to claim 1, wherein the head portion is connected to the tail portion through a cyclic amidine moiety.

6. Compound according to claim 4, which is selected from the group of compounds of formulas 1 to 6.

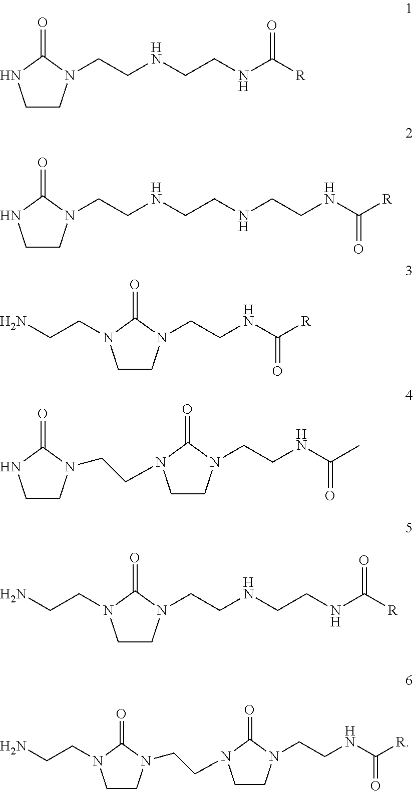

7. Compound according to claim 5, which is selected from the group of compounds of formulas 7 and 8.

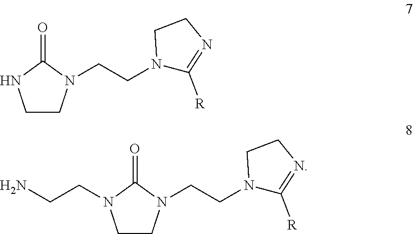

8. Compound according to claim 1, wherein the tail portion comprises a straight-chain alkyl or alkenyl groups with about 6-about 20 carbon atoms, which may be substituted with groups selected from one to three C1-C3 alkyl groups, one to three hydroxyl groups, and one or two carboxylic acid groups.

11. Compound according to claim 1, wherein the tail portion is the tail of a fatty acid selected from the group octanoic acid, nonanoic acid, decanoic acid, undecanoic acid, undecylenic acid, dodecanoic acid, tridecanoic acid, tetradecanoic acid, hexadecanoic acid, octadecanoic acid, oleic acid, and mixtures thereof.

12. Process for inhibiting corrosion of a surface susceptible thereto, wherein the surface is contacted with an anticorrosive compound, the compound being a compound according to claim 1.

13. (canceled)

14. Compound according to claim 1, wherein the head portion comprises about 3 to about 6 alkylene moieties present between nitrogen atoms.

15. Compound according to claim 2, wherein the alkylene moieties are selected from propylene moieties, isopropylene moieties and ethylene moieties.

16. Compound according to claim 2 wherein the head portion is connected to the tail portion through an amide moiety.

17. Compound according to claim 3 wherein the head portion is connected to the tail portion through an amide moiety.

18. Compound according to claim 2, wherein the head portion is connected to the tail portion through a cyclic amidine moiety.

19. Compound according to claim 3, wherein the head portion is connected to the tail portion through a cyclic amidine moiety.

20. Compound according to claim 4, which is selected from the group of compounds of formulas 1 to 6, wherein R is a substituted alkyl or alkenyl group with about 6-about 24 carbon atoms:

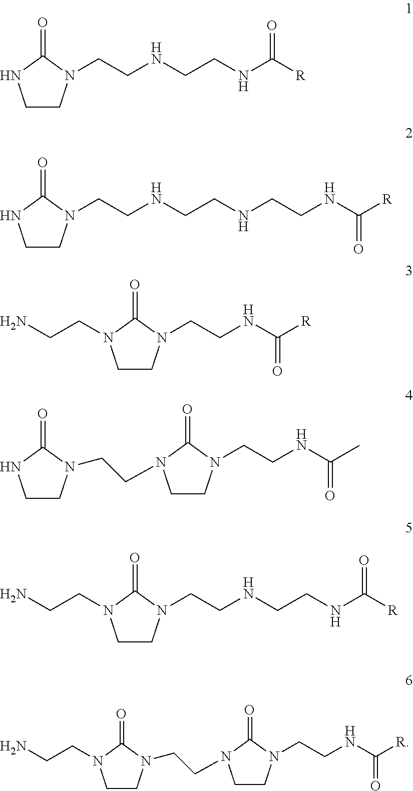

21. Compound according to claim 5, which is selected from the group of compounds of formulas 7 and 8, wherein R is a substituted alkyl or alkenyl group with about 6-about 24 carbon atoms. [chemical structure]
